(19) United States
(12) Patent Application Publication
(10) Pub. No.: US 2007/0142326 A1
(43) Pub. Date: Jun. 21, 2007

(54) Treatment of a condition in a mammal with administration of aminosugar and uses thereof

(76) Inventor: Youe-Kong SHUE, Carlsbad, CA (US)

Correspondence Address: CATALYST LAW GROUP, APC, 9710 SCRANTON ROAD, SUITE S-170, SAN DIEGO, CA, 92121, US

(21) Appl. No.: 10/574,054

(22) PCT Filed: Sep. 30, 2004
(86) PCT No.: PCT/US04/32048

§ 371 Date: Jun. 7, 2006

(51) Int. Cl. A61K 31/7008; A61K 9/14

(52) U.S. Cl. 514/62; 424/489; 424/423

(57) ABSTRACT

The present invention relates to treating joint related conditions in mammals by administering an aminosugar, and wherein said treatment specifically prevents, lessens or reverses pathologies associated with the joint condition, said pathologies being selected from the group consisting of synovitis, subchondral bone edema, and cartilage degradation.

### RELATED APPLICATIONS

This application claims priority from Provisional Patent Application No. 60/507,716 filed on Oct. 1, 2003, entitled TREATMENT OF A CONDITION IN A MAMMAL WITH ADMINISTRATION OF AMINOSUGAR AND USES THEREOF.

### GOVERNMENT GRANTS

This invention was made in part with United States government support under grant number NIH AG 07996 and AT 00052 awarded by the National Institutes of Health. The U.S. Government may have certain rights in this invention.

### FIELD OF THE INVENTION

This invention relates to methods of treating the severity of joint related conditions in mammals by administering an aminosugar and wherein said treatment specifically prevents, lessen or reverse many of the pathological markers associated with said joint conditions, said pathological markers being selected from the group consisting of synovitis, subchondral bone edema, and cartilage degradation.

### BACKGROUND OF TEE INVENTION

A variety of pathological markers are associated with joint related conditions and diseases. A joint related condition or disease can include, but is not limited to physical injury to the joint, osteoarthritis (OA) and rheumatoid arthritis. In particular, the associated pathological markers can include synovitis, subchondral bone edema, and progressive cartilage degradation, although numerous others do exist. (Ayral et. al. Rheumatology, Vol 35, 14-17; McAlindon, Best Pract Res Clin Rheumatol 1999; 13(2):329-44; Ayral et. al. Annals of the Rheumatic Diseases 2002 vol. 61 suppl. 1, #OP0014; Kerin et. al. Cell Mol Life Sci 2002; 59:27-35; Hedbom et. al. Cell Mol Life Sci 2002; 59:45-53; Silver et. al. Crit Rev Biomed Eng 2001; 29:373-91; Elsaid et al. Osteoartritis Cartilage. 2003; 11:673-80; Altman et al. Am J Med. 1983 Oct. 31; 75(4B):50-5).

Unfortunately, current therapies for joint conditions are typically limited to acetaminophens, non-steroidal anti-inflammatory drugs, injectable intra-articular corticosteroids, and hyaluronic acid, which only treat general inflammation and offer some level of pain relief. (Geletka et al. Best Pract Res Clin Rheumatol. 2003 October; 17:791-809; Altman et. al. Am J Med. 1983 Oct. 31; 75(4B):50-5).

More recently, glucosamine (GlcN) is used for treating the joint condition osteoarthritis, and its mechanism of action has been generally accepted to be as an anti-inflammatory. Only recently has there been suggestion that GlcN may halt progressive joint space narrowing to improve the biomechanics of osteoarthritic knee joints (Reginster et al. Lancet 2001; 357:251-6; Hughes et. al. Rheumatology (Oxford) 2002; 41:279-84). Unfortunately, the optimal in vitro activity for GlcN is observed at a concentration range that is difficult or even impossible to achieve with the orally administered sugar, and additionally, GlcN is cytotoxic for chondrocytes at low millimolar concentrations, making treatment with high concentrations of GlcN undesirable. (Sandy et. al. Arch Biochem Biophys 1999; 367:258-64; Shikhman et. al. J Immunol 2001; 166:5155-60; Setnikar et. al. Arzneimittelforschung 2001; 51:699-725; Adebowale et. al. Biopharm Drug Dispos 2002; 23:217-25; Aghazadeh-Habashi et. al. J Pharm Sci 2002; 5(2):181-4; de Mattei et. al. Osteoarthritis Cartilage 2002; 10:816-25.).

N-acetylglucosamine (GlcNAc) has also been utilized for treating OA and RA. Studies have shown that GlcNAc, in contrast to GlcN, is not toxic to human articular chondrocytes and does not induce chondrocyte cell death even at concentrations as higher than 50 mM. (de Mattei et. al. Osteoarthritis Cartilage 2002; 10:816-25.).

A number of patents relate to the use of GlcN and GlcNAc for the treatment of OA and/or RA. U.S. Pat. No. 3,683,076 (Rovati) discloses the use of GlcN salts for the treatment of OA and RA; U.S. Pat. No. 4,870,061 (Speck) discloses the use of GlcNAc for treating degenerative joint diseases via buccal administration; U.S. Pat. No. 5,840,715 and U.S. Pat. No. 6,136,795 (both Florio) disclose the use of GlcNAc as a nutritional supplement in dietary regime to provide relief from arthritis.

However, to date, there has been no effective means to treat, prevent, or lessen the severity of synovitis, subchondral bone edema, and cartilage degradation. Accordingly, there remains a great need for treatment, prevention, and lessening of the severity of these pathologies associated with conditions of the joint.

### SUMMARY OF THE INVENTION

It is discovered that aminosugars lessen, prevent and reverse the pathologies associated with joint conditions. This discovery, therefore, provides for the specific treatment of any condition of the joint wherein one or more of these pathologies are present. This newly discovered method of treating joint conditions having one or more of said pathological markers is a targeted approach, wherein the newly discovered affects of aminosugars allow for treatment of joint conditions that would otherwise have not received aminosugar therapy.

The present invention relates to treating joint related conditions in mammals by administering an aminosugar, and wherein said treatment specifically prevents, lessens or reverses pathologies associated with the joint condition, said pathologies being selected from the group consisting of synovitis, subchondral bone edema, and cartilage degradation.

A preferred embodiment of the present invention relates to methods of preventing, lessening or reversing the severity pathologies associated with joint conditions by administering to a mammal a therapeutically effective amount of an aminosugar including, but not limited to N-acetylglucosamine, glucosamine, galactosamine, N-acetylgalactosamine, iminocyclitol, and pharmaceutically acceptable salts thereof. In one aspect of this preferred embodiment, a joint condition is evaluated for specific pathological markers, and if said markers are present, a therapeutically effective amount of an aminosugar is administered. In an alternative aspect of the preferred embodiment, joint conditions know in the art to have these pathological markers associated therewith are treated using a therapeutically effective amount of an aminosugar. Preferably, the therapeutically effective amount of an aminosugar is intra-articularly administered to a mammal. Also preferably, that aminosugar is GlcNAc and more preferably that aminosugar is GlcNAc contained in a matrix as a controlled release formulation.

In another preferred embodiment of the present invention, GlcNAc is intra-articularly administered to a mammal having a joint condition to treat cartilage degeneration, subchondral bone edema and synovitis. Preferably, the treatment affects are seen at the macroscopic level and the microscopic level. Also preferably, the treatment effects are particularly the retardation of cartilage degeneration, the reduction of hypercellularity in marrow of subchondral bone edema and the reduction of membrane inflammation for synovitis.

Another preferred embodiment of the present invention is methods for administering to a mammal a composition comprising a therapeutically effective amount of an aminosugar, preferably GlcNAc, either alone or in combination with an existing anti-inflammatory drug or a hexoaminidase inhibitor. Preferably, methods for administering formulations of the present invention include, but are not limited to, intra-articular, topical, and intra-muscular methods. More preferably, controlled release formulations of the aminosugar are intra-articularly administered to mammals in need of such treatment.

Thus, the current invention provides methods for specifically treating joint conditions having one or more of the pathological markers that respond favorably to aminosugar therapy. The current invention also provides new uses for aminosugars in the targeted treatment of joint conditions having one or more of the pathological markers. The current invention furthermore provides compounds and pharmaceutical formulations thereof that are useful for the targeted treatment of joint conditions having one or more of the pathological markers.

### BRIEF DESCRIPTION OF THE DRAWINGS

FIG. 1A shows the gross morphological grading of femoral condyles in rabbits with bilateral anterior cruciate ligament transection (ACLT) and treated with intramuscular GlcNAc or normal saline.

FIG. 1B shows the gross morphological grading of tibial plateau in rabbits with bilateral anterior cruciate ligament (ACL) transection and treated with intra-muscular GlcNAc or normal saline.

FIG. 2 shows the gross morphological grading of femoral condyles in rabbits with unilateral ACL transection and treated with intra-articular GlcNAc, Sodium hyaluronate or saline.

FIG. 3 shows the gross morphological grading of tibial plateaus in rabbits with unilateral ACL transection and treated with intra-articular GlcNAc, Sodium hyaluronate or saline.

FIG. 4 illustrates the gross morphological assessment of joint swelling in rabbits with unilateral ACL transection and treated with intra-articular GlcNAc, Sodium hyaluronate or saline.

FIG. 5 illustrates DNA content in synovial tissue from rabbits with unilateral ACL transection and treated with intra-articular GlcNAc, Sodium hyaluronate or saline.

FIG. 6 shows the digital image analysis of the lesion size in femoral condyles (FIG. 6A) and tibial plateaus (FIG. 6B) from rabbits with unilateral ACL transection and treated with intra-articular GlcNAc or Sodium hyaluronate.

FIG. 7 shows the time dependant in vitro release of GlcNAc entrapped in injectable polymeric formulations according to one embodiment of the present inventions.

### DETAILED DESCRIPTION OF THE INVENTION

Abbreviations and Terms

In accordance with the present invention and as used herein, the following terms and abbreviations are defined with the following meanings, unless explicitly stated otherwise. These explanations are intended to be exemplary only. They are not intended to limit the terms as they are described or referred to throughout the specification. Rather, these explanations are meant to include any additional aspects and/or examples of the terms as described and claimed herein.

The following abbreviations are used herein:

ACL=Anterior Cruciate Ligament;

ACLT==Anterior Cruciate Ligament Transection

GlcN=glucosamine;

GAGs=glycosaminoglycans;

GlcNAc=N-Acetylglucosamine;

HA=hyaluronic acid;

IL-1β=interleukin-1β;

IL-6=interleukin-6;

NSAID=nonsteroidal anti-inflammatory drug;

OA=osteoarthritis;

PBS=phosphate-buffered saline;

PEG=polyethylene glycol;

PMSF=phenylmethylsulfonyl fluoride; and

RA=rheumatoid arthritis;

The term “active ingredient” refers to a therapeutically effective amount of drug or formulation thereof. Preferably, active ingredients of the present invention are aminosugars, more preferably the aminosugars GlcNAc and GlcN; and most preferably is the aminosugar GlcNAc.

The term “therapeutically effective amount” refers to the amount of an active ingredient necessary to induce one or more of the desired pharmacological effects of the current invention. The amount can vary greatly according to the effectiveness of a particular active substance; the age, weight, and response of the individual; as well as the nature and severity of the individual's symptoms. Accordingly, there is no upper or lower critical limitation with respect to the amount of the active substance. A therapeutically effective amount to be employed in the present invention can readily be determined by those skilled in the art.

The term “alginate gel” refers to natural polysaccharide polymers comprising 1,4-linked β-D-mannuronic and α-L-guluronic acid residues in varying proportions. Alginate is capable of forming stable gels, particularly in the presence of certain divalent cations, such as calcium, barium, and strontium.

The term “aminosugar” refers to any synthetic or naturally occurring sugar wherein one or more carbon atoms are substituted with an amino group (—NH₂). Such substitution may occur without regard to orientation or configuration of any asymmetric carbons present in the sugar. Unless stated otherwise, the term “aminosugar” refers to either anomer (α or β) of a cyclic aminosugar. Aminosugars may be N-substituted with alkyl or acyl group, where one hydrogen atom of a pendant amino group is replaced by an alkyl or acyl moiety (—COR where R=lower alkyl). According to one preferred embodiment of the invention, R in —COR=methyl (—CH₃).

The term “arthritis” refers to any particular disease characterized by joint inflammation, although the etiology of the inflammation may differ in various conditions. Relatively common arthritic diseases include rheumatoid arthritis, juvenile arthritis, ankylosing spondylitis, psoriatic arthritis and osteoarthritis.

The terms “articular cartilage” or “cartilage” refer to a substance that covers ends of bones and forms the joint surfaces. Cartilage can withstand compressive forces and creates a low friction surface upon which the joints can glide. Articular cartilage comprise chondrocytes and a substrate further comprising proteins and glycosaminoglycan polysaccharides.

The term “cartilage degradation” refers to degradation in the tissues comprising cartilage.

The term “chitin” refers to (poly)GlcNAc linked in a β-1,4 fashion. Chitin is found throughout nature, for example in the exoskeletons of insects and crustacea.

The term “chitosan” refers to deacylated chitin or (poly)N-glucosamine linked in a β-1.4 fashion.

The term “chondrocyte” refers to cells found within articular cartilage. Chondrocytes produce collagen, a gelatinous protein, and proteoglycans, which are glucosamine glycans linked to proteins (also called mucopolysaccharides).

The term “condition of the joint” or “joint condition” means any disease affecting the joint of a mammal and which presents with one or more of the following pathological conditions: synovitis, subchondral bone edema, and cartilage degeneration.

The term “encapsulation efficiency” refers to the amount of a compound or active ingredient encompassed, incorporated, loaded, associated, bound or otherwise entrapped within injectable polymeric gels, liposomes, microspheres, nanoparticles, or the like. In general, “yield” is expressed as a percent encapsulation of the active ingredient.

The term “entrapped” or “encapsulated” refers to any method of formulating an active ingredient, which confines, sequesters, or otherwise inhibits the free dissolution of the active ingredient in a matrix, such as a solution or solid phase. Preferred examples of entrapping or encapsulating active ingredients include, but are not limited to, formulations entrapped in a matrix wherein said matrix is selected from a particle, an implant, or a gel.

The term “matrix” refers to a solid, gel or liquid composition capable to entrapping an aminosugar(s), and optional additional materials, such as an anti-inflammatory drug, therein.

The term “glycosaminoglycan” refers to long heteropolysaccharide molecules containing repeating disaccharide units. The disaccharide units may comprise modified aminosugars: D-, N-acetylgalactosamine or D-GlcNAc and an uronic acid such as D-glucuronate or L-iduronate. Among other functions, GAGs serve as a lubricating fluid in the joints. Specific GAGs of physiological significance are hyaluronic acid, dermatan sulfate, chondroitin sulfate, heprin, heparan sulfate, and keratan sulfate.

The term “hyaluronic acid” refers to a naturally occurring mucopolysaccharide comprising alternating subunits of D-glucuronic acid and D-N-acetyl glucosamine. Hyaluronic acid is a linear polysaccharide (long-chain biological polymer) formed by repeating disaccharide units consisting of D-glucuronic acid β(1-3) N-acetyl-D-glucosamine linked by β(1-4) glycosidic linkages. Hyaluronic acid is commercially available in several molecular weight ranges spanning from about 50,000 Daltons to about 8×10.sup.6 Daltons. Hyaluronic acid is also available as a sodium salt and is a dried, highly purified substance. Sodium hyaluronate may be preserved with a variety of preservatives known in the art including, but not limited to, alkyl-substituted benzoic acid esters, alcohols, conjugates, blends, and mixtures thereof.

The term “hyaluronan” refers to a polymer of repeating molecules of N-acetylglucosamine and glucuronic acid.

The term “IL-1β” refers to interleukin-1β, an immunomodulator that mediates a wide range of immune and inflammatory responses, including the activation of B- and T-cells.

The term “injectable formulation” refers to a sterile, injectable composition prepared as a liquid solution or suspension. Solid forms suitable for solution in, or suspension in, liquid vehicles prior to injection may also be prepared. The preparation may also be emulsified or the active ingredient entrapped. An injectable formulation may also comprise a variety of preservatives known in the art, including, but not limited to, alkyl-substituted benzoic acid esters, alcohols, conjugates, blends, and mixtures thereof.

The term “injectable polymer gel” refers to a polymeric matrix carrier used to entrap or encapsulate active ingredients of the invention. Polymer-based injectable formulations allow drug dosage and timing to be tailored through the choice and formulation of various active ingredient/polymer combinations. The total dose of medication and the kinetics of release are variables that can be adjusted. For example, by varying the solvent content, copolymer ratio and molecular weight, and polymer solvent polarity drug delivery parameters can be optimized. Polymer-based systems may also increase the life span of active ingredients. The use of polymeric systems comprising poly lactide and lactide-glycolide copolymers in formulations offers certain advantages such as biocompatability and biodegradability. Injectable polymer gels may be prepared, e.g., processed, mixed, filtered, heated, or sterilized according to processes known in the art.

The term “microsphere” refers to a polymeric matrix carrier used to entrap or encapsulate active ingredients of the invention. Microsphere-based formulations allow drug dosage and timing to be tailored through the choice and formulation of various active ingredient/polymer combinations. The total dose of medication and the kinetics of release are variables that can be adjusted. For example, by varying the copolymer ratio and copolymer molecular weight, drug delivery parameters can be optimized. Microsphere-based systems may also increase the life span of active ingredients. The use of microspheres comprising lactide-glycolide copolymers in formulations offers certain advantages such as biocompatability and biodegradability. Microspheres may be prepared, e.g., processed, machined, milled, ground, or extruded according to processes known in the art.

The term “intra-articular” refers to a method of delivering a drug directly to a joint. Traditional routes of drug delivery, such as for example, oral, intravenous or intramuscular administration, depend upon vascular perfusion of the synovium to carry the drug to the joint. This is inefficient because transynovial transfer of small molecules from the synovial capillaries to the joint space generally occurs by passive diffusion, which becomes less efficient with increasing size of the target molecule. Thus, the access of directing molecules, for example, GlcN, to the joint space is substantially restricted. Intra-articular injection or perfusion of drugs circumvents those limitations.

The term “polymeric” refers to hyaluronic acid, polyethylene glycol, copolymers of polyethylene glycol and poly(lactic/glycolic acid), polymers of lactic acid, and copolymers of poly (ethylene glycol-y-(DL-lactic acid-co-glycolic acid), alginate gels, chitosans, or pharmaceutically acceptable salts thereof.

The term “sustained release” refers to the time period during which a drug is released for availability, or otherwise becomes available for physiological uptake. Periods of sustained release may be preceded by an induction period, during which little or no drug is released, or may be biphasic, comprising an initial time period during which some drug is released, and a second time period during which additional drug is released. In contrast, the term “continuous release” is used solely to describe a release profile that appears to be monophasic, having a smooth-curved time profile of release. Those of skill in the art will appreciate that the release profile may actually correspond to an exponential or logarithmic time-release profile.

The term “synovitis” means inflammation of the joint lining (synovium). Synovitis is present in a variety of joint related conditions, including, but not limited to osteoarthritis, physical or traumatic injury, rheumatoid arthritis and other autoimmune disorders.

It is discovered that aminosugars lessen, prevent and reverse the pathologies associated with joint conditions. This discovery, therefore, provides for the specific treatment of any condition of the joint wherein one or more of these pathologies are present. This newly discovered method of treating joint conditions having one or more of said pathological markers is a targeted approach, wherein the newly discovered affects of aminosugars allow for treatment of joint conditions that would otherwise have not received aminosugar therapy. Note that the term “pathology”, “pathologies” and “pathological markers” are used interchangeably herein, however, their reference is to synovitis, subchondral bone edema, and cartilage degradation.

The present invention relates to treating joint related conditions in mammals by administering an aminosugar, and wherein said treatment specifically prevents, lessens or reverses pathologies associated with the joint condition, said pathologies being selected from the group consisting of synovitis, subchondral bone edema, and cartilage degradation.

A preferred embodiment of the present invention relates to methods of preventing, lessening or reversing the severity pathologies associated with joint conditions by administering to a mammal a therapeutically effective amount of an aminosugar including, but not limited to N-acetylglucosamine, glucosamine, galactosamine, N-acetylgalactosamine, iminocyclitol, and pharmaceutically acceptable salts thereof. In one aspect of this preferred embodiment, a joint condition is evaluated for specific pathological markers, and if said markers are present, a therapeutically effective amount of an aminosugar is administered. In an alternative aspect of the preferred embodiment, joint conditions know in the art to have these pathological markers associated therewith are treated using a therapeutically effective amount of an aminosugar. Preferably, the therapeutically effective amount of an aminosugar is intra-articularly administered to a mammal. Also preferably, that aminosugar is GlcNAc and more preferably that aminosugar is GlcNAc contained in a matrix as a controlled release formulation.

In another preferred embodiment of the present invention, GlcNAc is intra-articularly administered to a mammal having a joint condition to treat cartilage degeneration, subchondral bone edema and synovitis. Preferably, the treatment affects are seen at the macroscopic level and the microscopic level. Also preferably, the treatment effects are particularly the retardation of cartilage degeneration, the reduction of hypercellularity in marrow of subchondral bone edema and the reduction of membrane inflammation for synovitis.

Another preferred embodiment of the present invention is methods for administering to a mammal a composition comprising a therapeutically effective amount of an aminosugar, preferably GlcNAc, either alone or in combination with an existing anti-inflammatory drug or a hexoaminidase inhibitor. Preferably, methods for administering formulations of the present invention include, but are not limited to, intra-articular, topical, and intramuscular methods. More preferably, controlled release formulations of the aminosugar are intra-articularly administered to mammals in need of such treatment.

Thus, the current invention provides methods for specifically treating joint conditions having one or more of the pathological markers that respond favorably to aminosugar therapy. The current invention also provides new uses for aminosugars in the targeted treatment of joint conditions having one or more of the pathological markers. The current invention furthermore provides compounds and pharmaceutical formulations thereof that are useful for the targeted treatment of joint conditions having one or more of the pathological markers.

All patents, publications and patent applications cited herein are hereby incorporated by reference in their entireties. Unless otherwise defined, all technical and scientific terms used herein have the same meaning as commonly understood by those of skill in the art to which this invention belongs. Exemplary methods and materials are described below. However, methods and materials similar or equivalent to those described herein can be also used to obtain variations of the present invention. The materials, methods, and examples are illustrative only and not intended to be limiting.

The following examples are provided by way of describing specific embodiments of the present invention without intending to limit the scope of the invention in any way.

The ACLT (anterior cruciate ligament transection) model of post-traumatic joint degeneration is one of the most widely used models for studying degenerative changes in articular cartilage. ACLT is described in Setton et. al. Osteoarthritis Cartilage 1999; 7:2-14. ACLT results in abnormal knee biomechanics including increased anterior drawer at extension and at 90o of flexion, as well as an increased internal rotation similar to that observed in human knees plagued with a joint condition, including traumatic injury or arthritis.

Osteoarthritis is only one of many joint conditions that presents with one or more of the following pathologies, synovitis, subchondral bone edema and cartilage degradation. Other joint conditions include, but are not limited to physical or traumatic injury and rheumatoid arthritis. As used herein below, and in conjunction with the discussion of the ACLT model, the term “experimental OA” does not limit the current invention to osteoarthritis. Rather, “experimental OA” is merely common nomenclature in the art. The invention is useful for the full range of joint conditions associated with the above mentioned pathologies.

In order to study the effects aminosugars have on the pathological markers associated with joint conditions, experimental OA was induced in the knee of rabbits by ACLT. The most severe areas of cartilage degeneration in these rabbits occurs in the medial femoral condyles followed by lateral femoral condyles (Chang et. al. Osteoarthritis Cartilage 1997; September; 5:357-72.). In the tibial plateaus ACL transection causes only mild to moderate lesions in the areas covered by the menisci.

Reagents. GlcNAc was purchased from Sigma (St. Louis, Mo.). GlcNAc was dissolved in normal saline and sterilized by filtration through 0.22 micrometer filter (Corning, Acton, Mass.). Sterile solution of GlcNAc was stored at 4oC. Sodium hyaluronate (Hyalgan™) was purchased from Sanofi-Synthelabo (New York, N.Y.).

Preparation of GlcNAc sustained release formulations.

Poly Lactic Acid Depot (PLAD). Lyophilized cGMP grade GlcNAc (Greenfield Inc, Gumee, Ill., USA) powders were dissolved or suspended in polymer solutions comprised of medical grade low molecular weight Poly-Lactic Acid (L-102, Boehringer Ingelheim (BI) Chemicals, Inc. Wallingford, Conn., USA) dissolved in USP/NF grade solvents (benzyl alcohol (BA), benzyl benzoate (BB), ethanol (EtOH)). The resulting mixtures were evaluated in vitro and in vivo.

Poly Lactic-co-Glycolic Acid (PLGA) injectable gels. Lyophilized GlcNAc powders were dissolved or suspended in polymer solutions comprised of medical grade low molecular wt. PLGA (RG 502-H, Boehringer Ingelheim (BI) Chemicals, Inc. Wallingford, Conn., USA) dissolved in USP/NF grade solvents (NMP, DMSO, benzyl alcohol, benzyl benzoate, ethanol). The resulting mixtures were evaluated in vitro and in vivo.

| Formulation | Composition | Amount (lot#) | Catalogue # |
|---|---|---|---|
| PLAD 1 | 20% PLA | 2.5 g BI (200479) | R202H |
| | 75% BB | 3.75 g Sigma (97H1569) | B9550 |
| | 5% BA | 0.25 g Sigma (11K3682) | B-1042 |
| PLAD 2 | 20% PLA | 2.5 g BI (200479) | R202H |
| | 79% BB | 3.95 g Sigma (97H1569) | B9550 |
| | 1% BA | 0.05 g Sigma (11K3682) | B-1042 |
| PLAD 3 | 20% PLA | 2.5 g BI (200479) | R202H |
| | 75% BB | 3.75 g Sigma (97H1569) | B9550 |
| | 5% NMP | 0.25 g Aldrich (01948CA) | 270458 |
| PLAD 4 | 20% PLA | 2.5 g BI (200479) | R202H |
| | 79% BB | 3.95 g Sigma (97H1569) | B9550 |
| | 1% NMP | 0.05 g Aldrich (01948CA) | 270458 |
| NMP 1 | 50% 502H | 1.0 g BI (1005122) | RG502H |
| | 50% NMP | 1.0 g Aldrich (01948CA) | 270458 |
| NMP 2 | 50% 503H | 1.0 g BI (1006370) | R503H |
| | 50% NMP | 1.0 g Aldrich (01948CA) | |
| NMP 3 | 50% 503H | 2.5 g BI (1006370) | R503H |
| | 50% NMP | 2.5 g Aldrich (01948CA) | 270458 |
| NMP 4 | 55% PLA | 2.75 g BI (200479) | R202H |
| | 45% NMP | 2.25 g Aldrich (01948CA) | 270458 |
| NMP 5 | 55% 502H | 2.75 g BI (1005122) | RG502H |
| | 45% NMP | 2.25 g Aldrich (01948CA) | 270458 |
| NMP 6 | 55% 503H | 2.75 g BI (1006370) | R503H |
| | 45% NMP | 2.25 g Aldrich (01948CA) | 270458 |
| NMP 7 | 25% PLA | 1.25 g BI (200479) | R202H |
| | 25% 502H | 1.25 g BI (1005122) | RG502H |
| | 50% NMP | 2.5 g Aldrich (01948CA) | 270458 |

Manufacturing and quality control. The injectable gel formulations were sterilized as aqueous solutions by terminal filtration through a 0.22 micron filter then dried aseptically. The sterile polymer solution and sterile GlcNAc powder were mixed using aseptic techniques at the point of use. The identity, purity, potency, sterility, and loading of each formulation were recorded in production batch records. HPLC or FT-IR were used to measure identity, purity, potency, and loading. Sterility was determined using a modified USP sterility test. Briefly, the sample was dissolved in an appropriate solvent (usually DMSO) then serially diluted with sterile water to level where any solvents present were no longer bacteriostatic. These diluted samples were submitted to the standard USP sterility test (USP monograph number<71>).

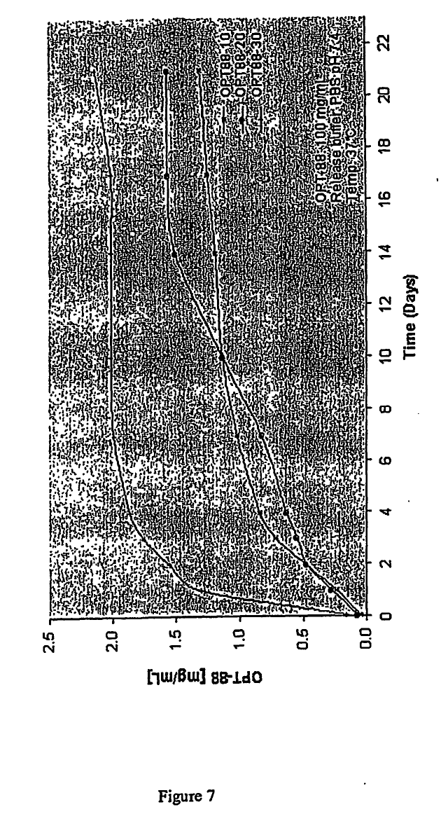

Release of GlcNAc from formulations incubated in phosphate buffered saline. An in vitro release study was performed in phosphate buffered saline to evaluated release kinetics under physiological conditions. Each sustained release formulation (100 μl) was placed in 1.0 mL of phosphate buffered saline and then incubated at 37° C. At different time points, the formulation matrix was separated by removing the phosphate buffered saline bathing solution with a pipet. The resulting solutions were analyzed by UV detection after GlcNAc derivatives were formed by methods previously described (Reissig et. al. J. Biol. Chem. 1955 217: 959-966). The formulation matrix was then re-suspended in 1.0 mL phosphate buffered saline and incubated under the same conditions as described above. The results are shown in FIG. 7, below.

Animals. New Zealand White rabbits, age 8-12 months, weight 3.7-4.2 kg, and with closed epiphyses, were used in all experiments except for the alzet pump study discussed below wherein the rabbits weighed 3.0-3.5 kg. All studies were performed in accordance with AACL guidelines and on approval of the animal review committees at both the University of California, San Diego and the Scripps Research Institute.

Anterior cruciate ligament transection (ACLT). Unilateral or bilateral ACLT was performed as indicated for each set of experiments. ACLT was performed using a medial arthrotomy technique (Yoshioka et al. Osteoarthritis Cartilage 1996; 4:87-98). After dislocating the patella laterally, the ACL was transected with a sharp blade. Complete transection was confirmed by a manual anterior drawer test The knee joints were irrigated with sterile saline and closed in layers with sutures. All animals were maintained individually with ad libitum activity. The animals were sacrificed 8 weeks after the surgery. Previously published data demonstrated that the majority of rabbits with ACLT develop cartilage degeneration at this time point (Sah et. al. J Orthop Res 1997 March; 15:197-203).

Intra-muscular injections of GlcNAc. Intra-muscular injections of GlcNAc were performed three times per week starting one week postoperatively for a period of seven weeks. The dose of GlcNAc was 200 mg/kg per injection. The control group of received the same number of intra-muscular injections of normal saline.

Intra-articular injections of GlcNAc. Intra-articular injections of GlcNAc started one week post-operatively for a period of seven weeks. Rabbits were injected twice per week with GlcNAc in a volume of 0.3 ml per knee joint The single dose of GlcNAc per injection was 80 mg. Control animals were injected twice per week intra-articularly with normal saline (0.3 ml per joint). The third group of rabbits received intra-articular injections of hyaluronan (0.3 ml per joint) twice per week for seven weeks starting one week after the ACL transection. Synovial fluid analysis was performed in 3 animals that developed gross synovial effusions (2 animals in the control group and 1 animal in the hyaluronan group). In all three animals synovial fluid was culture negative.

Gross morphological assessment of the knee joints. Gross morphological assessment of the knee joints included assessment of joint swelling, synovial effusion, macroscopic articular cartilage morphology of tibial plateaus and femoral condyles, and assessment of the menisci.

The following grading system was used to assess joint swelling: Grade 0-normal; Grade 1 (mild swelling)—mild inflammation and/or proliferation of the joint capsule; Grade 2 (moderate swelling)—thickening of joint capsule and/or inflammation of the synovium; Grade 3 (severe swelling)—abundant inflammation of the synovium, swelling of the menisci or ligaments (anterior cruciate ligament or posterior cruciate ligament).

The following grading system was used to assess synovial effusions: Grade 0—normal; Grade 1 (mild effusion)—effusion is greater than normal, but does not fill the knee joint; Grade 2 (moderate effusion)—effusion fills the knee joint, but does not pour out of the capsule as it is opened; Grade 3 (severe effusion)—effusion expands the knee joint and pours out as the capsule is opened.

Gross morphological assessment of the articular cartilage. The distal femur and proximal tibia were harvested keeping a 3.5 cm to 4 cm shaft of the bones. The articular cartilage surface of each specimen was covered with a solution consisting of India ink (Eberhard Faber, Lewisburg, Tenn.) in PBS (1:5 ratio). Excess ink solution was removed by gentle blotting with a tissue that was pre-moistened with PBS. Subsequently, all joints were photographed and digital images were analyzed.

The following grading system was used for articular cartilage assessment: Grade 1 (intact surface)—surface is normal in appearance and does not retain India ink; Grade 2 (minimal fibrillation)—surface retains India ink as elongated specks or light gray patches; Grade 3 (overt fibrillation)—areas which are velvety in appearance and retain India ink as intense black patches; Grade 4 (erosion)—loss of cartilage exposing the underlying bone.

Digital imaging. Articular surfaces of femoral condyles and tibial plateaus were gently blotted dry and cleaned of loose tissue. Each femoral shaft was clamped to an optical bench. An image (resolution: 60 pixels per mm; onscreen magnification: 20×) of the femoral condyles was obtained using a Canon EOS D30 digital camera with a 100 mm macro lens at a distance of approximately 12 cm. A millimeter scale was included in the photograph to accurately scale the image. The scaled image was then projected onto a 3D model of the femoral condyles. The 3D surface area of the lesion was measured by interactively plotting the margins of the lesion. A digital image of articular surface of the tibia was obtained as described above. No 3D projection was used since the tibial surface was relatively flat and 2D measurements do not vary significantly from 3D measurements.

Histological grading of the knee joints. Distal femur and proximal tibia from the rabbit knee joints were fixed in 10% buffered formalin, decalcified in TBD-2 decalcifier (ThermoShandon, Pittsburg, Calif.) and embedded in paraffin blocks. Sagittal sections of lateral and medial femoral condyles, and coronal sections of tibial plateaus were used for further histological analysis.

The assessment of sulfated glycosaminoglycan (SGAG) content was performed after staining of the tissue sections with Safranin O/Fast Green.

The following grading system was used for assessing SGAG content: Grade 1—Less than 25% loss of Safranin O staining, Grade 2—25-50% loss of Safranin O staining; Grade 3—More than 50% loss of Safranin O staining;

The following grading system was used for assessing cartilage integrity: Grade 1—Intact cartilage surface; Grade 2—Presence of fibrillations; Grade 3—Full thickness cartilage defect. In addition, all tissue samples were analyzed for the presence of chondrocyte proliferation or cloning.

Histological assessment of synovium was based on the presence of synovial proliferation and synovial neoangiogenesis, and it was performed separately for the synovium attached to the tibial plateaus, lateral, and medial femoral condyles.

Microscopic assessment of bone marrow was based on the presence of subchondral bone marrow hypercellularity and increased vascularization as shown in Table 3.

Measurement of DNA content in synovial tissue. Synovial tissue cellularity was assessed by quantitating the tissue concentration of DNA (Amiel et. al. J Orthop Res 1986; 4:162-172). Briefly, washed and lyophilized synovial tissue was solubilized by incubation for 2 hours in 1 N NaOH at 65° C. Duplicate aliquots were reacted with 0.04% indole-HCl reagent and mixed with chloroform to remove interfering substances. The aqueous phase containing the DNA was harvested, and the absorbance was measured at 490 nm. Calf thymus DNA was used as a standard. Results were expressed as mg DNA per mg of dry tissue.

Statistical analysis of experimental data was performed using Microsoft's Excel Analysis ToolPak.

Alzet Pump Administration of GlcNAc. New Zealand White rabbits, 3.0-3.5 kg, were used in this experiment. They were randomly allocated into five groups, each group having 8 rabbits. Group A was treated with saline (negative control group); Group B was treated with 1.5 M GlcNAc group; Group C was treated with 0.5 M GlcNAc group; Group D was treated with 0.15 M GlcNAc group; Group E was treated with 0.05 M GlcNAc group. All compounds were continuously delivered to the joints by Alzet mini pumps. The delivery rate for this pump was 2.5 μl/hour. All the rabbits received ACLT surgery on the right knee and GlcNAc was delivered to the right knee.

GlcNAc was administered to the right knee by alzet pump for 8 weeks starting immediately following ACLT procedure. The pumps were replaced at the end of week 4. Pump and delivery tube were checked twice a week to make sure that the delivery tube remained in place at the joint. At the end of experiment, pictures were taken with a digital camera to demonstrate that the polyethylene tubing (ID: 0.58 mm) was still inside the joint.

The gross morphological changes of both knee, including joint swelling and joint fluid, were evaluated. The distal femur and proximal tibia of each operated and contra lateral control knee were harvested. The occurrence, site and severity of lesions on the surface of the samples were determined by established criteria under light microscope. The India ink stained femoral condyles and tibial plateaus were also photographed with a digital camera. The surface areas of the stained lesions on digital images were quantified and compared between each group by image analysis software.

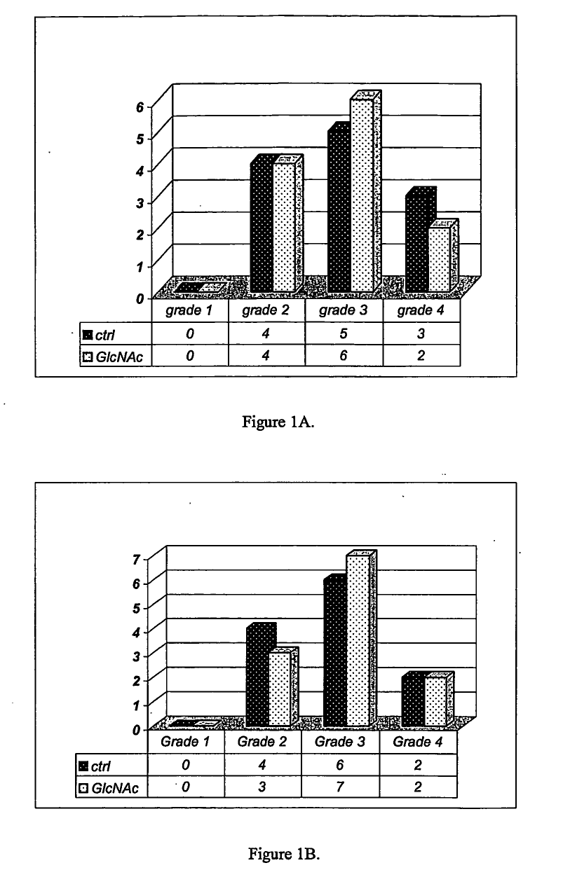

The efficacy of intramuscular GlcNAc was assessed in 6 rabbits having bilateral ACLT in compare to 6 control rabbits also having bilateral ACLT, but having received only intramuscular injections of saline. Gross morphological analysis of the tibial plateaus and femoral condyles did not reveal a statistically significant difference in the degree of cartilage damage between the treatment and the control populations. FIGS. 1a and 1b are plots of the gross morphological assessment of these groups. FIG. 1a plots the assessment of the femoral condyles, while FIG. 1b plots the assessment of the tibial plateaus.

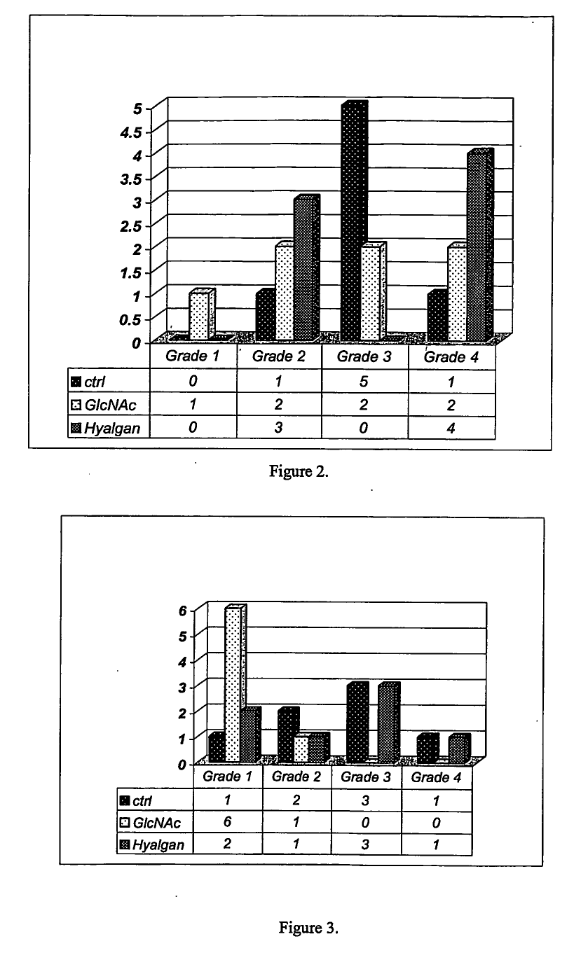

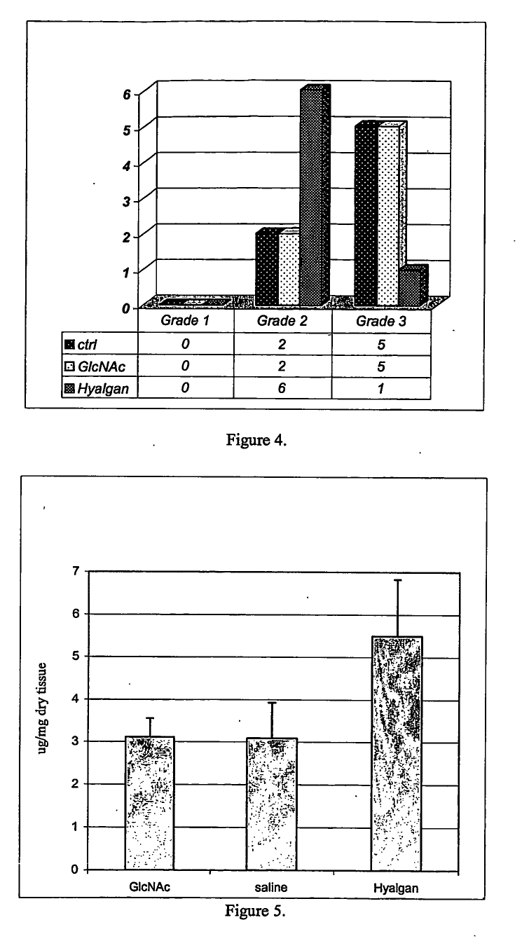

Intra-articular injection of GlcNAc, on the other hand, shows improvement in the condition of the tibial plateaus and femoral condyles. Rabbits having bilateral ACLT were injected intra-articularly with either GlcNAc (treatment group, n=7) or saline (control group, n=7) two times per week for a total of seven weeks. As seen in FIG. 2, gross morphological analysis of the femoral condyles demonstrated a trend towards improved cartilage condition (improved lesions) for the treatment group over the control group. Furthermore, as is shown in FIG. 3, morphological analysis of the tibial plateaus revealed remarkable chondroprotective activity of GlcNAc in that only 1 of 7 treatment rabbits developed a cartilage lesion compared to 6 of 7 in the control group developing such lesions (p<0.003). FIGS. 4 and 5 show that intra-articular administration GlcNAc does not significantly affect joint swelling, synovial effusions or DNA content in synovial tissue.

Thus, intra-muscular injection of GlcNAc does not demonstrate chondroprotective effects, but does show a trend towards reduction of synovitis. Intra-articular administration of GlcNAc, however, does show a significant reduction of cartilage degradation at both the macroscopic and microscopic levels.

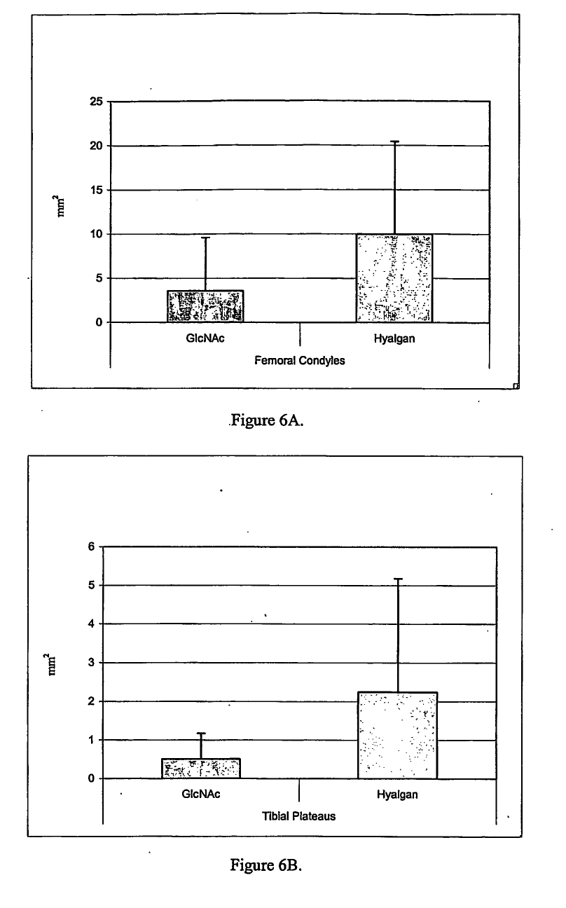

A comparison study of intra-articularly administered GlcNAc and intra-articularly administered hyaluronan was then performed. As discussed above, preparations of hyaluronan are commonly used as viscosupplementation for treating knee osteoarthritis. In this study, gross morphological analysis of the femoral condyles shows no significant difference between the GlcNAc group and the hyaluronan group (n=7). FIG. 2. However, as is seen in FIG. 3, GlcNAc shows significantly greater (p<0.01) chondroprotective activity than does hyaluronan. Furthermore, the surface areas of the cartilage lesions are greatly reduced in the GlcNAc group as compared to the hyaluronan group. FIGS. 6a and 6b. There is no significant difference in synovial effusion between the GlcNAc group and the Hyaluronan group (FIG. 4); however, DNA content assessment reveals a significant reduction in synovial hyperplasia and cellularity (p<0.05) for the GlcNAc group over the hyaluronan group (FIG. 5).

A histological analysis of experimental rabbits receiving either GlcNAc treatment or saline was performed and the results from each population were compared. As is seen in table 1 below, there is a similar loss of SGAG in the medial femoral condyles for both the treatment population and the control population. However, analysis and comparison of SGAG at the tibial plateaus shows a trend towards improvement for the GlcNAc group, and furthermore, there is seen a significantly reduced loss of SGAG in the lateral femoral condyles for this group. Examination of the cartilage integrity for these groups, also demonstrates significant chondroprotective activity on the tibial plateaus and lateral femoral condyles for the GlcNAc group over the control group.

A histological assessment of synovitis demonstrates that GlcNAc suppresses synovial proliferation (table 2). The effect of GlcNAc on synovial neoangiogenesis shows regional variability; however, GlcNAc shows significant improvement adjacent to the medial femoral condyles. Table 2. Examination of the subchondral bone marrow shows that GlcNAc treatment reduces hypercellularity and capillary dilution leading to subchondral bone edema. Table 3.

Collectively, histological analysis of the cartilage, synovium and subchondral bone marrow/edema demonstrates the chondroprotective and anti-inflammatory effects of intra-articularly administered GlcNAc.

TABLE 1
Histological grading of articular cartilage

| | | | |
|---|---|---|---|
| | | Intra-articular | |
| | Saline | GlcNAc | |
| Anatomical area | N = 7 | N = 6 | P value |

Sulfated glycosaminoglycan loss

| | | | |
|---|---|---|---|
| Medial femoral condyle | 1.57 ± 0.53 | 1.66 ± .33 | P ≦ 0.4 |
| Lateral femoral condyle | 2.43 ± 0.53 | 1.50 ± 0.83 | P ≦ 0.03 |
| Tibial plateau | 1.71 ± 0.76 | 1.16 ± 0.40 | P ≦ 0.14 |

Impairment of cartilage surface integrity

| | | | |
|---|---|---|---|
| Medial femoral condyle | 1.66 ± 0.41 | 1.66 ± 0.81 | P ≦ 0.91 |
| Lateral femoral condyle | 2.00 ± 1.00 | 1.33 ± 0.51 | P ≦ 0.17 |
| Tibial plateau | 1.71 ± 0.49 | 1.16 ± 0.41 | P ≦ 0.05 |

TABLE 2
Histological grading of synovium

| | | | |
|---|---|---|---|
| | | Intra-articular | |
| | Saline | GlcNAc | |
| Anatomical area | N = 7 | N = 6 | P value |

Synovial thickening/proliferation

| | | | |
|---|---|---|---|
| Medial femoral condyle | 7/7 | 2/6 | P ≦ 0.006 |
| Lateral femoral condyle | 7/7 | 2/6 | P ≦ 0.006 |
| Tibial plateau | 7/7 | 1/6 | P ≦ 0.0002 |

Synovial neoangiogenesis

| | | | |
|---|---|---|---|
| Medial femoral condyle | 5/7 | 1/6 | P ≦ 0.05 |
| Lateral femoral condyle | 4/7 | 1/6 | P ≦ 0.16 |
| Tibial plateau | 4/7 | 1/6 | P ≦ 0.16 |

TABLE 3
Histological grading of subchondral bone marrow

| | Saline | Intra-articular GlcNAc | |
|---|---|---|---|
| Anatomical area | N = 7 | N = 6 | P value |
| Medial femoral condyle | 3/7 | 0/6 | P ≦ 0.07 |
| Lateral femoral condyle | 3/7 | 0/6 | P ≦ 0.07 |
| Tibial plateau | 3/7 | 1/6 | P ≦ 0.35 |

These results show that intra-articular administration of GlcNAc significantly and unexpectedly reduced cartilage degradation as measured by macroscopic and microscopic criteria. Moreover, maximal chondroprotective activity was observed in tibial plateaus followed by lateral femoral condyles and medial femoral condyles, strongly indicating that the joint areas with less severe cartilage destruction are more sensitive to therapeutic effects of GlcNAc. Finally, intra-articular GlcNAc administration unexpectedly reduced the severity of synovitis and also improved hypercellularity of the subchondral bone marrow. Furthermore, GlcNAc can be delivered to the joint continuously for more than three weeks after a single administration (intra-articular) as determined using the sustained release and alzet pump methods described above.

Upon intra-articular administration of GlcNAc, the maximal chondroprotective activity was observed in tibial plateaus followed by lateral femoral condyles and, finally, medial femoral condyles, indicating that the joint areas prone to minimal degeneration do respond to GlcNAc administration better than the areas with severe cartilage damage. In compare, the intra-muscular administration of GlcNAc in rabbits with experimental OA did not demonstrate chondroprotective benefits but did reveal significant reduction of synovitis; an anti-inflammatory activity. Intra-articular administration of GlcNAc was much more potent than intramuscular administration. Rabbits treated with intra-articular GlcNAc demonstrated significant retardation of cartilage degeneration on both macroscopic and microscopic levels. Finally, intra-articular GlcNAc is superior to visco-supplementation therapy with hyaluronan in regard to their chondroprotective efficacy.

In summary, intra-articular therapy of experimental OA rabbits with GlcNAc unexpectedly reduced cartilage degradation with a macroscopic reduction in lesion size in mammals, significantly suppressed synovitis, and reduced the bone marrow hypercellularity of subchondral bone edema.

### Pharmaceutical Formulation and Administration

Once isolated, an aminosugar, most preferably, GlcNAc, as the active ingredient, can be put in pharmaceutically acceptable formulations, such as those described in Remington's Pharmaceutical Sciences, 18th ed., Mack Publishing Co., Easton, Pa. (1990), incorporated by reference herein, and used for specific treatment of diseases and pathological conditions with little or no effect on healthy tissues. The preparation of a pharmacological composition comprising active ingredients dissolved or dispersed therein need not be limited based on formulation. Such compositions may be prepared as injectable liquid solutions or suspensions. However, solid forms suitable for dissolution, or resuspension, in liquid prior to use can also be prepared. The preparation can also be emulsified.

In a preferred embodiment, the composition is held within a container, which includes a label stating to the effect that the composition is approved by the FDA in the United States (or other equivalent labels in other countries) for treating a disease or condition described herein. Such a container will provide therapeutically effective amount of the active ingredient to be administered to a host.

The particular aminosugars that affect the conditions of interest can be administered to a mammal either by alone or in pharmaceutical compositions where it is mixed with suitable carrier(s) or excipient(s). In treating a mammal exhibiting a condition of interest, a therapeutically effective amount of an agent or agents, such as GlcNAc, is administered. The active ingredient can be mixed with excipients that are pharmaceutically acceptable and compatible with said active ingredient and in amounts suitable for use in the therapeutic methods described herein.

Pharmaceutically acceptable salts can be prepared by standard techniques. For example, the free base form of the compound is first dissolved in a suitable solvent such as an aqueous or aqueous-alcohol solution, containing the appropriate acid. The salt is then isolated by evaporating the solution. In another example, the salt is prepared by reacting the free base and acid in an organic solvent.

Carriers or excipients can be used to facilitate administration of the compound, for example, to increase the solubility of the compound. Examples of carriers and excipients include calcium carbonate, calcium phosphate, various sugars or types of starch, cellulose derivatives, gelatin, vegetable oils, polyethylene glycols, water, saline, dextrose, glycerol, ethanol and physiologically compatible solvents.

Compositions of the present invention can include pharmaceutically acceptable salts of the components therein. Pharmaceutically acceptable salts include acid addition salts (formed with any free amino groups of the aminosugars) that are formed with inorganic acids such as, for example, hydrochloric or phosphoric, sulfuric acids, etc., or such organic acids as acetic, tartaric, mandelic and the like. Salts formed with the free carboxyl groups of the aminosugars can also be derived from inorganic bases such as, for example, sodium, potassium, ammonium, calcium or ferric hydroxides, and such organic bases as isopropylamine, trimethylamine, 2-aminoethanol, histidine, procaine and the like.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized.

For any aminosugar compound used in the methods of the invention, the therapeutically effective dose can be estimated initially from cell culture assays. For example, a dose can be formulated in animal models to achieve a circulating plasma concentration range that includes the IC50 as determined in cell culture (i.e., the concentration of the test compound which achieves a half-maximal disruption of the protein complex, or a half-maximal inhibition of the cellular level and/or activity of a complex component). Such information can be used to more accurately determine useful doses in humans. Levels in plasma may be measured, for example, by HPLC.

Another preferred embodiment of the present invention relates to an improved formulation for the active ingredient, GlcNAc. Encapsulation or entrapment of GlcNAc in liposomes or other entrapping agents modifies its pharmacodynamic profile when intra-articularly injected. Preferably, GlcNAc is entrapped in a matrix. More preferably, GlcNAc in entrapped in a matrix selected from the groups consisting of a particle, an implant, or a gel.

The exact formulation, route of administration and dosage can be chosen by the individual physician in view of the mammal's condition. (See e.g. Fingl et al., in The Pharmacological Basis of Therapeutics, 1975, Ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual mammal. A program comparable to that discussed above may be used in veterinary medicine.

Depending on the specific conditions being treated, such agents may be formulated and administered systemically or locally. Techniques for formulation and administration may be found in Remington's Pharmaceutical Sciences, 18th ed., Mack Publishing Co., Easton, Pa. (1990), which is incorporated herein by reference.

For injection, the agents of the invention may be formulated in aqueous solutions, preferably in physiologically compatible buffers such as Hanks's solution, Ringer's solution, or physiological saline buffer.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group.

It is to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

We claim:

1. A method of treating a pathology in a mammal, said pathology being selected from the group consisting of synovitis, subchondral bone edema, and cartilage degradation, and said treatment comprising administering to said mammal a therapeutically effective amount of an aminosugar.

2. The method according to claim 1, wherein said aminosugar is selected from the group consisting of N-acetylglucosamine, glucosamine, galactosamine, N-acetylgalactosamine, iminocyclitol, and pharmaceutically acceptable salts thereof.

3. The method according to claim 1, wherein said aminosugar is entrapped in a matrix.

4. The method according to claim 3, wherein said matrix is selected from the group consisting of a particle, an implant, or a gel.

5. The method according to claim 4, wherein said particle comprises a liposome, a nanosphere, a microsphere, or a suspension.

6. The method according to claim 4, wherein said implant comprises a polymer, a pump, or a device.

7. The method according to claim 4, wherein said gel comprises an in situ implant forming gel, a semi-solid gel, a hydrogel, or a thermo sensitive gel.

8. An injectable formulation for intra-articular treatment of a pathologies associated with a joint condition comprising an aminosugar which is entrapped by a matrix, wherein said matrix comprises a particle, an implant, or a gel.

9. A method of treating pathologies associated with a joint condition comprising administering a therapeutically effective amount of N-acetylglucosamine as a controlled release formulation.

10. The method according to claim 9, wherein N-acetylglucosamine is administered by intramuscular injection or intra-articular injection.

11. The method according to claim 9, wherein N-acetylglucosamine is administered by subcutaneous injection or infusion.

12. The method of claim 9 wherein the pathologies associated with a joint condition is selected from the group consisting of synovitis, subchondral bone edema and cartilage degradation.

13. The method of claim 12 wherein the pathologies is synovitis.

14. The method of claim 12 wherein the pathologies is subchondral bone edema.

15. The method of claim 12 wherein the pathologies is cartilage degradation.

16. The method of claim 9 wherein the joint condition is not osteoarthritis.

17. The method of claim 9 wherein the joint condition is not rheumatoid arthritis.

18. A method of treating a joint condition comprising the steps of:
a. diagnosing a pathological marker associated with a joint condition; and
b. administering an aminosugar in a therapeutically effective formulation.

19. The method of claim 18 wherein the pathological marker is selected from the group consisting of synovitis, subchondral bone edema and cartilage degradation.

20. The method of claim 19 wherein the pathological marker is synovitis.

21. The method of claim 19 wherein the pathological marker is subchondral bone edema.

22. The method of claim 19 wherein the pathological marker is cartilage degredation.

23. The method of claim 18 wherein the joint condition is not osteoarthritis.

24. The method of claim 18 wherein the joint condition is not rheumatoid arthritis.

25. The method of claim 18 wherein the step of administering an aminosugar is performed by an administration route selected from the group consisting of intra-articular, intramuscular, infusion pump or subcutaneous.

26. The method of claim 25 wherein the administration route is intra-articular.

27. The method of claim 18, wherein the aminosugar is selected from the group consisting of N-acetylglucosamine, glucosamine, galactosamine, N-acetylgalactosamine, iminocyclitol, combination therapies thereof, pharmaceutically acceptable salts thereof, and injectable formulations thereof.

28. The method of claim 27 wherein the aminosugar is N-acetylglucosamine.

29. The method of claim 27 wherein the injectable formulations thereof are selected from the group consisting of matrix particle, matrix gel and controlled release formulation.

30. The method of claim 27 wherein the combination therapy thereof combined the aminosugar with a compound selected from the group consisting of anti-inflammatory drugs and hexoaminidase inhibitors.

31. A method of preventing cartilage degradation comprising administering in mammals who have less severe said cartilage degradation comprising a therapeutically effective amount of N-acetylglucosamine as a controlled release formulation.

32. The method according to claim 31, wherein said N-acetylglucosamine is administered by intra-muscular or intra-articular injection.

33. The method according to claim 21, wherein said N-acetylglucosamine is administered by subcutaneous injection or infusion pump.
